(19) United States
(12) Patent Application Publication
(10) Pub. No.: US 2004/0137069 A1
(43) Pub. Date: Jul. 15, 2004

(54) Pirenzepine ophthalmic gel

(76) Inventor: Harun Takruri, Newport Beach, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/698,320

(22) Filed: Oct. 31, 2003

Related U.S. Application Data
(63) Continuation of application No. PCT/US02/13823, filed on May 1, 2002.
(60) Provisional application No. 60/293,731, filed on May 25, 2001.

(51) Int. Cl. A61K 9/14

(52) U.S. Cl. 424/488

(57) ABSTRACT

It is a primary object of the present invention to provide an aqueous ophthalmic formulation, for treating myopia, comprising pirenzepine in combination with a pharmaceutically acceptable gel carrier.

### RELATED APPLICATIONS

This application is a continuation and claims the benefit of priority of International Application No. PCT/US02/13823 filed May 1, 2002, designating the United States of America and published in English, which claims the benefit of priority of U.S. Provisional Application No. 60/293,731 filed May 25, 2001, both of which are hereby expressly incorporated by reference in their entireties.

### FIELD OF THE INVENTION

The present invention is in the field of aqueous ophthalmic pharmaceutical formulations.

### BACKGROUND OF THE INVENTION

Myopia, axial elongation of the eye, affects a large proportion of the population. Commonly, the onset of myopia is during the grade school years and progresses until growth of the eye is completed. A pharmacologic therapy which prevents or retards the developmental abnormality of myopia would represent. a major advance in the treatment of myopia.

The potential use for a pharmacologic therapy has been stimulated by evidence that atropine prevents the development of myopia in humans (D. A. Goss 1982 Am J Optom Physiol Opt 59:828-841.), tree shrews (McKanna J. A. & V. A. Casagrande 1978 Exp Eye Res 26:715-723.), stump-tailed monkeys and chicks (McBrien N. A. et al. 1991 Biochem Soc Trans 19:861-865; McBrien N. A. et al. 1991 Invest Ophthalmol Vis Sci 32:1203; Tigges M. et al. 1996 Invest Ophthalmol Vis Sci 37:S326.). The clinical use of atropine as a therapy has been limited due to its ocular side effects including glare from pupillary dilation and blurred vision due to loss of accommodation. Mild cycloplegic agents like tropicamide have been effective in a number of studies but failed in other studies (Curtin B. J. & D. B. Karlin 1971 Am J Ophthalmol 71:42-53.).

Stone and Laties found that subconjunctival injections of atropine, a nonselective muscarinic antagonist, and pirenzepine, a relatively selective M1-antagonist marketed for systemic use in Europe for its anti-dyspepsia properties, attenuated axial eye growth in a chick model of myopia. M2 and M3 antagonists did not prevent axial elongation (Stone R. A. et al. 1991 Exp Eye Res 52:755-758; U.S. Pat. No. 5,112,522, Filed May 11, 1990). Unlike atropine, a selected concentration of pirenzepine may prevent myopia without inducing unwanted side effects such as disabling mydriasis and cycloplegia.

Pirenzepine is a relatively selective M1-muscarinic antagonist. It is being investigated for its topical ocular use to moderate and halt the progression of pediatric myopia. Administered as a solution in up to 2% strength, it was found comfortable and without systemic effects in adult volunteers (Shedden A.H. et al. 1998 Invest Ophthalmol Vis Sci 39:S279.).

However, work on a solution dosage form of pirenzepine indicated a physical appearance problem. Pirenzepine is stable in solution especially at pH 5, but its degradation product is insoluble in water. Thus, the accumulation of even a small amount of degradation product over the shelf-life of the solution results in an unacceptable product due to the unattractive appearance of the precipitate in the solution.

There are no “standard” formulation solutions for such a problem. One approach is to use a refrigerated solution. Another approach is to use a lyophilized product for reconstitution prior to dispensing to the patient. However, neither of these approaches is optimal. Lyophilization adds considerably to the cost of the product and requires cumbersome reconstitution processes. Refrigeration is not always convenient. Thus, there is a need for pirenzepine in a dosage form that solves the physical appearance problem using a formulation approach that is deemed desirable.

### SUMMARY OF THE INVENTION

Accordingly, it is a primary object of the present invention to provide an aqueous ophthalmic formulation, for treating myopia, comprising pirenzepine in combination with a pharmaceutically acceptable gel carrier.

### DETAILED DESCRIPTION OF THE PREFERRED EMBODIMENT

The ophthalmic aqueous gel formulation of the present invention for treating myopia comprises a pharmaceutically effective amount of pirenzepine in combination with a water soluble cellulose derivative.

The concentration of the pirenzepine in the present formulation may range from about 0.001 to 3% (w/v), preferably about 0.005 to 2% (w/v). Pirenzepine and its dihydrochloride salt are known in the art.

Below is the structure of pirenzepine dihydrochloride:

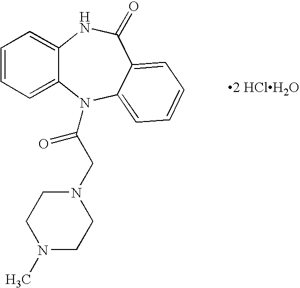

Molecular formula: C₁₉H₂₁N₅O₂.2 HC1.H₂O

Molecular weight: 442.3; 351.4 (anhydrous free base)

Chemical Names: 5,11-dihydro-11-[(4-methyl-1-piperazinyl)acetyl]-6H pyrido[2,3-b][1,4]-benzodiazepin-6-one dihydrochloride monohydrate 11-[(4-methyl-1-piperazinyl)acetyl]-pyrido[2,3-b][1,4]-benzodiazepin-6(5H)-one dihydrochloride monohydrate

Cellulose derivatives are used as gelling agents in the formulation of this invention. Most preferred is hydroxypropyl methylcellulose. Any cellulose derived gelling agent, however, that forms an aqueous gel at the desired viscosity, i.e., is soluble in water and forms a gel, can be used. Such derivatives are well known, as are their properties, and are described, e.g., in the U.S. Pharmacopeia (2000) (UNITED STATES PHARMACOPEIAL CONVENTION, INC., THE UNITED STATES PHARMACOPEIA/THE NATIONAL FORMULARY (2000)). Such gelling agents include, but are not limited to, methyl cellulose, hydroxypropyl cellulose, carboxymethyl cellulose, hydroxyethyl cellulose, and cellulose gum. Combinations of various derivatives may also be used. Cellulose based gelling agents are advantageous over, for example, cross-linked acrylic polymers. For example, Carbopol™, a cross-linked acrylic polymer, has been used to form an aqueous gel containing pilocarpine hydrochloride for ophthalmic use. Cellulose based gelling agents, however, are less likely to cause adverse reactions.

The formulations of the invention are substantially viscous enough to form a viscous gel. The viscosity preferably is in the range of 10,000 to 300,000 centipoise (cps), most preferably 15,000-200,000 cps, at about 20° C. and shear rate of 1s⁻¹ based on Brookfield RVDV analysis.

In the aqueous gel for ophthalmic use, the amount of cellulose based gelling agent is preferably from about 0.5 wt. % to 5 wt. %, most preferably from about 1 wt. % to 5 wt. %.

Suitable cellulose based preparations for use in the invention are commonly commercially available. For example, sources of hydroxypropyl methylcellulose that are suitable for making a cellulose based ophthalmic gel according to the invention include Ashland Distribution Co., Asiaamerica International Inc., Biddle Sawyer Corp., Carbomer Inc., Colorcon Inc., Dow Chemical Co., FOB Chemicals, Hercules Inc., Mutchler Inc., Penta Mfg Co., Spectrum Laboratory Products Inc., Van Waters & Rogers Inc., and Warner Jenkinson.

The formulation may contain additional pharmaceutically inactive substances. For example, it may contain one or more solubilizing agents, such as polysorbate 20, polysorbate 40, polysorbate 60, or polysorbate 80. The formulation may also contain a dispersant, such as lecithin or glycerin. Collagen can also be added. Other additives include cyclodextrins, in particular alpha, beta, and gamma cyclodextrins. Also, vitamin E, particularly in solubilized form, or other antioxidants, including butylate hydroxyanisole (BHA) and butylate hydroxytoluene (BHT), may be added. Some additional examples of inactives follow: sodium chloride, cetrimide, thimerosal, benzalkonium chloride, boric acid, sodium carbonate, potassium chloride, propylene glycol, polyoxyethylene, polyoxypropylene, polyoxyl 40 stereate, polyvinyl alcohol, poloxamer 188, sodium citrate, sodium thiosulfate, sodium bisulfite, dextran 70, acetic acid, polyethylene glycol, povidone, dextrose, magnesium chloride, alginic acid, sodium acetate, sodium borate, edetate disodium, sodium hydroxide, and hydrochloric acid. The optimal amount of inactive ingredient employed in the formulation can be conventionally determined based on the particular active pharmaceutical, and the intended use.

The formulation of the invention can be placed in any desired dispensing device suitable for an ophthalmic formulation. The device can be an ophthalmic delivery system such as a sterile ophthalmic tube, for example, a conventional 3.5-5 g tube having an ophthalmic tip and containing the ophthalmic formulation of the invention, or a sterile, single or daily use container containing 0.1-0.5 g of the formulation.

The pharmaceutical formulations can be administered via various routes including ocular instillation, subconjunctival administration, and intravitreal administration. A typical daily dose of pirenzepine may range 6 mg or less/whole body weight, preferably 4 mg or less/whole body weight, and can be administered in a single dose or in divided doses. However, it should be understood that the amount of pirenzepine actually administered ought to be determined in light of various relevant factors including the myopia to be treated, the chosen route of administration and the severity of the patient's symptom; and, therefore, the above dose should not be intended to limit the scope of the invention in any way.

The stability data generated on the gel and solution dosage forms show the superiority of the gel over the solution in maintaining an acceptable physical appearance in the presence of the small amounts of the water-insoluble degradation product referred to in the background of the invention.

The following Examples are intended to further illustrate the scope of the invention without limiting its scope.

### EXAMPLE 1

An aqueous ophthalmic gel of 2.0% pirenzepine for the treatment of myopia according to the present invention was prepared as follows:

TABLE 1
Pirenzepine ophthalmic gel formulations.

| | 0.5% | 1.0% | 2.0% |
|---|---|---|---|
| Ingredient | (in mg/g) | (in mg/g) | (in mg/g) |
| Pirenzepine dihydrochloride | 6.3 | 12.6 | 25.2 |
| (base equivalent) | (5.0) | (10.0) | (20.0) |
| Hydroxypropyl Methyl-Cellulose | 20 | 20 | 20 |
| (K100M, Dow Chemical Co.) | | | |
| Sodium acetate | 0.40 | 0.40 | 0.40 |
| Benzalkonium chloride | 0.05 | 0.05 | 0.05 |
| Edetate disodium | 0.15 | 0.15 | 0.15 |
| Sodium chloride | 5.0 | 3.5 | 0.0 |
| Sodium Hydroxide (q.s. to pH) | 5.0 | 5.0 | 5.0 |
| Purified Water, q.s. to | 1.00 g | 1.00 g | 1.00 g |

Part 1: Purified water was heated to 80-90° C. Hydroxypropyl methylcellulose (HPMC) was added and mixed until it was uniformly dispersed. The pH was adjusted to 5.0±1.0 with sodium hydroxide, but this was not a critical step and can be eliminated. After being placed in a pressure vessel, the mixture was sterilized at 121° C. for 30-45 minutes. In another embodiment, autoclaving is conducted under nitrogen when oxygen plays a role in viscosity loss upon autoclaving. The mixture was cooled to 25° to 30° C. and mixed for several hours to yield a homogenous viscous gel. Batches manufactured in the appropriate jacketed pressure vessel showed that chilling Part 1 (the hydroxypropyl methylcellulose phase) to about 10° C. rather than to 25° to 30° C. after autoclaving greatly enhanced the hydration and consequently the viscosity of the gel. The gel was stored at 25° to 30° C. for several hours to aid in dissolution and then maintained at 25° to 30° C. for storage.

Part 2: The rest of the ingredients were mixed and dissolved in water until a clear solution was obtained. The pH was adjusted to 5.0±1.0 with sodium hydroxide. The solution was sterilized by membrane filtration (0.2 microns).

The concentration of pirenzepine is calculated based on the free base. However, we added its dihydrochloride salt. By adjusting the pH to 5.0±1.0 with sodium hydroxide, the dihydrochloride salt is partially or completely converted to the monohydrochloride salt.

The solution of Part 2 was aseptically added to the gel of Part 1. Sufficient sterile water was added to q.s. to the final weight of the batch. A final pH adjustment was made, if necessary. The batch was mixed for about 48 hours to achieve homogeneity. A gel resulted that was used to aseptically fill pre-sterilized ophthalmic containers.

### EXAMPLE 2

The ophthalmic pirenzepine gel preparation made in Example 1 was administered as follows (the ophthalmic tip of the dispensing mechanism did not touch any surface to avoid contamination). The lower lid of the eye to be administered was pulled down and a small amount of gel (approximately 0.25 inches) was applied to the inside of the eyelid. The gel was applied to the afflicted eye twice per day. A gel formulation in a target population of pediatric subjects was well tolerated.

### EXAMPLE 3

Procedure for Viscosity Measurement: A Brookfield Cone and Plate Viscometer (Model RVDV-III+) was used to measure viscosity at about 20° C. and shear rate of 1s^{31 1}. The viscosities of 0.5-2 g samples of various gels were measured. Gels with viscosities of 5,000 to less than 600,000 cps were tested with a CP52 spindle, and other spindles are used depending on the viscosities of the gels.

While the present invention has been described in some detail for purposes of clarity and understanding, one skilled in the art will appreciate that various changes in form and detail can be made without departing from the true scope of the invention. All patents, applications, and publications, referred to above, are hereby incorporated by reference.

What is claimed is:

1. An aqueous ophthalmic gel formulation for the treatment of myopia comprising pirenzepine and an amount of gelling agent effective to form an aqueous gel, said gel having a Brookfield RVDV viscosity of from about 10,000 to about 300,000 cps at about 20° C. and sheer rate of 1s^{31 1}, wherein said gelling agent is a water soluble cellulose derivative.

2. A formulation according to claim 1 wherein the concentration of said pirenzepine is from about 0.001 to 3% (w/v).

3. A formulation according to claim 1 wherein the concentration of said pirenzepine is from about 0.005 to 2% (w/v).

4. A formulation according to claim 1 wherein said water soluble cellulose derivative is soluble in said aqueous formulation at a viscosity of about 15,000 to about 200,000 cps at about 20° C. and sheer rate of 1s^{31 1}.

5. A formulation according to claim 1 wherein said water soluble cellulose derivative is soluble in said aqueous formulation at a viscosity of about 100,000 cps at about 20° C. and sheer rate of 1s⁻¹.

6. A formulation according to claim 1 wherein said amount of gelling agent is an amount of from about 0.5 to 5 wt. %.

7. A formulation according to claim 1 wherein said amount of gelling agent is an amount of from about 1 to 5 wt %.

8. A formulation according to claim 1, further comprising at least one member selected from the group consisting of sodium chloride, cetrimide, thimerosal, benzalkonium chloride, boric acid, sodium carbonate, potassium chloride, propylene glycol, polyoxyethylene, polyoxypropylene, polyoxyl 40 stereate, polyvinyl alcohol, poloxamer 188, sodium citrate, sodium thiosulfate, sodium bisulfite, dextran 70, acetic acid, polyethylene glycol, povidone, dextrose, magnesium chloride, alginic acid, sodium acetate, sodium borate, edetate disodium, sodium hydroxide, and hydrochloric acid.

9. A formulation according to claim 1 wherein said gelling agent is at least one member selected from the group consisting of hydroxypropyl methylcellulose, methyl cellulose, hydroxypropyl cellulose, carboxymethyl cellulose, hydroxyethyl cellulose, and cellulose gum.

10. A formulation according to claim 1 wherein said gelling agent is hydroxypropyl methylcellulose.

11. An ophthalmic delivery system containing the formulation of claim 1.

12. The ophthalmic delivery system of claim 11 comprising an ophthalmic tube having an ophthalmic tip and containing said aqueous gel.

13. A method of treating myopia comprising administering the formulation of claim 1 to the eye of a human individual, whereby myopia is treated.

14. The method of claim 13 wherein said human individual is a pediatric subject.

15. A method of making the formulation of claim 1 comprising autoclaving a mixture comprised of said gelling agent and water, sterile filtering a solution comprising said pirenzepine and water, and aseptically admixing them.

16. The method of the claim 17 wherein said autoclaving step is conducted under nitrogen.

17. A formulation according to claim 1 wherein the formulation is selected from the group consisting of the formulations of Table 1.

18. A formulation according to claim 1 in sterile form.
